(19) United States
(12) Patent Application Publication
(10) Pub. No.: US 2019/0046449 A1
(43) Pub. Date: Feb. 14, 2019

(54) A UNIQUE HIGH-SHEAR GRANULATION PROCESS FOR IMPROVED BIOAVAILABILITY OF RIVAROXABAN

(71) Applicant: Mylan Inc., Canonsburg, PA (US)

(72) Inventors: Boyong Li, Morgantown, WV (US); Thomas Daniel Reynolds, Morgantown, WV (US)

(73) Assignee: Mylan Inc., Canonsburg, PA (US)

(21) Appl. No.: 16/076,454

(22) PCT Filed: Feb. 25, 2016
(86) PCT No.: PCT/US16/19543

§ 371 Date: Aug. 8, 2018

(51) Int. Cl. A61K 9/16; A61K 9/20; A61K 31/5377

(52) U.S. Cl. CPC A61K 9/1694; A61K 9/1623; A61K 9/1652; A61K 31/5377; A61K 9/2018; A61K 9/2054; A61K 9/2031; A61K 9/1641

(57) ABSTRACT

Method for preparing granules of rivaroxaban together with pharmaceutically acceptable excipients using high-shear mixing. The granules produced by the methods disclosed herein may be incorporated into dosage forms for oral administration having enhanced oral bioavailability of rivaroxaban.

### BACKGROUND OF THE INVENTION

### FIELD OF THE INVENTION

The present disclosure relates generally to the field of pharmaceutical sciences and more specifically to a process for the preparation of granules of rivaroxaban together with one or excipients for incorporation into pharmaceutical dosage forms.

### BACKGROUND OF THE INVENTION

Rivaroxaban, chemically known as (S)-5-chloro-N-{[2-oxo-3-[4-(3-oxomorpholin-4-yl)phenyl]oxazolidin-5-yl]methyl} thiophene-2-carboxamide, has the following chemical formula:

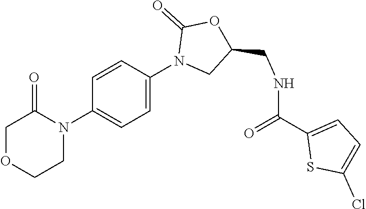 [SMILES: O=C(NC[C@H]1CN(c2ccc(N3CCOCC3=O)cc2)C(=O)O1)c1ccc(Cl)s1]

Rivaroxaban is an oral anticoagulant drug, a direct factor Xa inhibitor, and is marketed as XARELTO® in the United States by Janssen Pharmaceuticals, Inc. XARELTO® is indicated for use as treatment to reduce the risk of stroke and systemic embolism in patients with nonvalvular atrial fibrillation; for the treatment of deep vein thrombosis (DVT), pulmonary embolism (PE); for the reduction in the risk of recurrence of DVT and of PE; and for the prophylaxis of DVT, which may lead to PE in patients undergoing knee or hip replacement surgery.

U.S. Patent App. Pub. No. 2008/0026057, which is hereby incorporated by reference, discloses a process of preparing rivaroxaban granules using hydrophilized rivaroxaban.

Rivaroxaban has poor water solubility (7 mg/L) and is thus difficult to incorporate into oral dosage forms that provide sufficient bioavailability of rivaroxaban following oral administration. There is thus a need to provide an improved process for the preparation of dosage forms of rivaroxaban with improved bioavailability of the rivaroxaban active ingredient.

Two conventional methods of granulation include high-shear granulation and fluid bed granulation. High-shear granulation involves adding a binder solution to powder, often a mixture of API and one or more excipients, and granulating the resulting mixture with blending tools and a chopper. The powder agglomerates into larger granules, held together by the binder. Granules formed by high-shear granulation typically are dense and compact—properties that result in good flow characteristics, which may improve final processing of a pharmaceutical dosage form.

In fluid bed granulation, a liquid binder is sprayed onto a powder suspended on a fluid bed. Powder particles bind to each other to form granules. When the desired size of granule is achieved, the spraying process may be stopped and the liquid may be evaporated. In some cases, liquid that may have been trapped inside the granule also evaporates. This may leave a void and create pores. Thus, granules formed by this process may have a lower density when compared to granules achieved by high-shear granulation techniques. Fluid bed granulation, however, often results in smaller particle sizes and narrower particle size distributions, which may improve the overall quality of the final product.

The present invention provides a process for preparing granules containing rivaroxaban that may be incorporated into a final oral dosage form. The granules prepared using methods disclosed herein may have improved characteristics over granules prepared using conventional methods, such as those noted above.

### SUMMARY OF THE INVENTION

One aspect of the present invention provides high-density granules of rivaroxaban together with one or more pharmaceutically acceptable excipients. Within the context of the present invention, these granules, which may be produced using a high-shear granulation technique, may have a narrower particle size distribution when compared to granules typically achieved by conventional techniques. The granules of the present invention may also have a similar bioavailability to granules produced by fluid bed granulation. In some embodiments, at least 50% of the population of granules of the present invention has a diameter of less than 0.105 mm.

Another aspect of the present invention provides a process for the preparation of high-density granules of rivaroxaban together with one or more excipients that have a narrow particle-size distribution. The process may include the following steps:

a. dry mixing rivaroxaban and a pharmaceutically acceptable excipient in a high-shear mixer to form a dry mix blend;

b. adding a binder solution to the dry mix blend to form a granulating mixture;

c. mixing the granulating mixture in a high-shear mixer to form the rivaroxaban-containing granules; and

d. drying and milling the rivaroxaban-containing granules.

The binder solution may include a binder dissolved in a solvent. The binder may be hypromellose, cellulose or cellulose derivatives, povidone, starch, sucrose, polyethylene glycol, or mixtures thereof. The solvent used in the binder solution may be water, C1-6 alcohol, or mixtures thereof.

The pharmaceutically acceptable excipient used in the methods of the present invention may be a lubricant, a glidant, a disintegrant, a bulking agent, a rate-controlling polymer, a filler, a surfactant, or mixtures thereof. In certain embodiments, the granulating mixture has a water content of less than 30% and the mixing step is carried out for between three minutes and five minutes.

Within the context of the present invention, the rivaroxaban-containing granules may be further combined with one or more pharmaceutically acceptable excipients and incorporated into an oral dosage form.

### DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 shows dissolution profiles of tablets prepared according to Examples 1 and 2; and

FIG. 2 shows dissolution profile of tablets prepared by Example 5 compared to XARELTO®.

### DETAILED DESCRIPTION OF THE INVENTION

It is to be understood that the description of the present invention has been simplified to illustrate elements That are relevant for a clear understanding of the invention.

The present invention provides an improved process for the preparation of granules of rivaroxaban together with one or more pharmaceutically acceptable excipients. The rivaroxaban-containing particles of the present invention may be incorporated into pharmaceutical dosage forms for oral administration to patients in need thereof.

In certain embodiments of the present invention, the granules are prepared using a low-moisture, high-shear granulation process with an extended mixing time. Granules prepared using the methods of the present invention are dense and finely granulated, and also have a narrow particle size distribution, as discussed further hereinbelow. These properties may result in enhanced granule consistency, improved workability of the final blend prior to tablet formation, and may also enhance the dissolution of the final dosage form and bioavailability of rivaroxaban. Surprisingly, when incorporated into oral dosage forms, these granules provide high oral bioavailability of the rivaroxaban API when compared to tablets formulated with granules prepared using conventional, prior-art high-shear granulation techniques.

Another aspect of the present invention provides a process for the preparation of granules of rivaroxaban together with one or more excipients, which may include the following steps:

a. dry mixing rivaroxaban and at least one pharmaceutically acceptable excipient in a high-shear mixer to form a dry mix blend;

b. adding a binder solution to the dry mix blend to form a granulating mixture;

c. mixing the granulating mixture in a high-shear mixer to form the rivaroxaban-containing granules; and

d. drying and milling the rivaroxaban-containing granules.

According to the present invention, rivaroxaban and one or more pharmaceutically acceptable excipients may be subjected to high-shear mixing in a high-shear mixer to form a dry mix blend. The rivaroxaban used in this step may be of any polymorphic form and may be present in a neutral form or as a pharmaceutically acceptable salt. Examples of pharmaceutically acceptable excipients suitable for use in this step include fillers, bulking agents, disintegrants, surfactants, lubricants, glidants, rate-controlling polymers, and mixtures thereof. One of skill in the art will recognize a variety of useful and appropriate excipients that may be mixed with rivaroxaban to form a dry mix blend. In certain embodiments, combining rivaroxaban with a mixture of microcrystalline cellulose, lactose monohydrate, croscarmellose sodium, and sodium lauryl sulfate resulted in a particularly useful dry mix blend. In certain embodiments of the present invention, the dry mix blend of rivaroxaban and one or more pharmaceutically acceptable excipients is substantially free of water.

According to the present invention, a binder solution may then be added to the dry mix blend of rivaroxaban and one or more pharmaceutically acceptable excipients, resulting in a granulation mixture. In particularly useful embodiments of the present invention, the binder solution is gradually added to the blend while the blend is subjected to high-shear granulation. Within the context of the present invention, the binder may be, for example, hypromellose, cellulose or cellulose derivatives, povidone, starch, sucrose, polyethylene glycol, or mixtures thereof. The binder may be dissolved in a solvent, which may be, for example, water, an alcohol, or mixtures thereof. In some embodiments, the alcohol is a C₁₋₆ alcohol. In particularly useful embodiments of the present invention, the binder solution is hypromellose dissolved in water.

Within the context of the present invention, a relationship exists between the moisture level of the granulation mixture and the duration of high-shear mixing employed to obtain the desired granules. Generally, because the granulation mixtures of the present invention have low moisture content, longer durations of high-shear mixing will be useful. By way of example, traditional high-shear granulation of rivaroxaban (see Example 2 below) utilizes a granulation mixture that possesses a moisture content of 34%. This traditional high-shear granulation process employs a granulation time after addition of the binder solution of approximately one minute. In contrast, when the moisture content is reduced to about 30%, a longer high-shear mixing time after addition of the binder solution may be employed to obtain the desired granule size and size distribution (see Example 5 below). In some embodiments of the present invention, when the moisture content of the granulation mixture is about 30% or less, the high-shear granulation times may range from about 3 minute to about 5 minutes.

As used herein, the term “about” means 10% above or below the value recited.

According to the present invention, the granules may then be dried and milled. Within the context of the present, the drying step may be achieved by well-known methods, for example, by oven tray drying, or by fluid bed drying. Milling may also be carried out by methods well known in the art, for example, by using a Fitzmill or a Comil.

In some embodiments, the particle size distribution may be used to characterize the granules produced by the processes of the present invention. The particle size distribution of the granules may be determined by passing the granules through a series stacked screens with decreasing mesh sizes and measuring the percent of granules that are too large to pass through the mesh of a particular screen. In some embodiments of the present invention, more than about 50% of the granules are smaller than 0.105 mm (i.e., will fall through a size #140 mesh screen). In some embodiments of the present invention, granules size distribution of granules produced by the methods disclosed herein may have the particle size distribution as shown in the following table for two embodiments.

| | Embodiment 1: | Embodiment 2: |
|---|---|---|
| | % of particles | % of particles |
| Screen Mesh Size | remaining | remaining |
| #40 | 8.7% | 5.5% |
| #60 | 17.9% | 15.3% |
| #80 | 8.3% | 8.5% |
| #100 | 4.8% | 1.7% |
| #140 | 5.6% | 6.4% |
| Left in Pan | 54.8% | 62.6% |
| (smaller than #140) | | |

Within the context of the present invention, the granules may be further included in a final dosage form, for example, into a tablet. In certain embodiments, inclusion of the granules into a final dosage form may be accomplished by adding one more pharmaceutically acceptable excipients to the granules. Examples of pharmaceutically acceptable excipients that may be used include lubricants, glidants, disintegrants, surfactants, fillers, bulking agents, rate-controlling polymers, and mixtures thereof. Examples of useful excipients include magnesium stearate, talc, silicon dioxide, magnesium carbonate, fumed silica, croscarmellose sodium, povidone, sodium starch glycolate, sodium stearate fumarate, cellulose and cellulose derivatives (e.g., microcrystalline cellulose, hydroxypropyl cellulose), lactose, calcium phosphate dibasic, mannitol, sucrose, crospovidone, sodium lauryl sulfate, polaxomer, polyoxyethylene sorbitan, fatty acid esters, hypromellose, hydrogenated vegetable oil, and mixtures thereof. In certain embodiments of the present invention, it has been found that that adding croscarmellose sodium and magnesium stearate is particularly useful for incorporating the granules into a final dosage form.

Tablets containing the granules of the present invention may also include a coating which may contain, for example, lactose monohydrate, colloidal silicon dioxide, carnuba wax, triacetin, hypromellose, polyethylene glycol (for example, polyethylene glycol 3350), titanium dioxide, iron oxide red, iron oxide yellow, polyvinyl alcohol, lecithin, talc, artificial colors and flavorings, and mixtures thereof.

Within the context of the present invention, tablets of rivaroxaban may contain from about 2.5 mg to about 20 mg rivaroxaban. In certain embodiments, tablets containing 2.5 mg, 5 mg, 7.5 mg, 10 mg, 15 mg, or 20 mg of rivaroxaban were found to be particularly useful. In some embodiments of the present invention, tablets also contain about 3.0% w/w hypromellose, about 38.6% w/w microcrystalline cellulose, about 27.2% w/w lactose monohydrate, about 5% w/w croscarmellose sodium, about 0.5% w/w sodium lauryl sulfate, about 0.5% w/w magnesium stearate, and about 0.25% w/w yellow iron oxide. In other embodiments of the present invention, tablets also contain about 3.0% w/w hypromellose, about 50.3% w/w microcrystalline cellulose, about 27.2% w/w lactose monohydrate, about 5% w/w croscarmellose sodium, about 1% w/w sodium lauryl sulfate, and about 1% w/w magnesium stearate.

The dissolution profile of tablets containing granules prepared using the methods disclosed herein may be measured by submerging tablets in a pH 4.5 acetate buffer with 0.2% sodium lauryl sulfate using the paddle method (USP Apparatus 2) at 75 rpm. The dissolution rate of certain embodiments of the present invention was found to be similar to that of the branded product, XARELTO®, as shown in the examples below.

The bioavailability of tablets formulated with the high-density finely granulated granules of the present invention was also compared with XARELTO®. As detailed in examples below, tablets including the high-density, finely granulated granules of the present invention have similar Cₘₐₓ values and have the same bioavailability (as measured by AUC_{inf}) as XARELTO® under both fasting and fed conditions.

High-shear granulation is a high-energy process that, under typical conditions, results in larger, high-density granules. It is surprising that by lowering the moisture content of the mixture in combination with increasing the high-shear mixing time results in smaller, high-density particles with a narrow particle size distribution. It is similarly surprising that the granules of the present invention that are prepared by high-shear granulation (a process that typically leads to oral dosage forms with low bioavailability of the API) may be a component of pharmaceutical oral dosage forms having a bioavailability of the API similar to tablets made from fluidized bed granules and of the branded product, XARELTO®. These unique characteristics of the API-containing granules of the present invention improve final processing as well as lead to a more reproducible and high-quality final product.

In view of the above description and the examples below, one of ordinary skill in the art will be able to practice the invention as claimed without undue experimentation. The foregoing will be better understood with reference to the following examples that detail certain procedures for the preparation of molecules, compositions, and formulations according to the present invention. All references made to these examples are for the purposes of illustration. The following examples should not be considered exhaustive, but merely illustrative of only a few of the many aspects and embodiments contemplated by the present disclosure.

### EXAMPLES

### Example 1

### Formulation of Tablets Prepared From Granules Prepared Using Fluidized Bed Granulation

Hypromellose was dissolved in purified water and sprayed into a fluidized mixture of rivaroxaban, lactose monohydrate, microcrystalline cellulose, croscarmellose sodium, and sodium lauryl sulfate in a fluid bed with top-spray system. Then, the dried granules were milled through a Fitzmill with #1A screen followed by blending with the extra-granular components in a blender. The resulting blend was compressed into tablets on a rotary press.

### Example 2

### Formulation of Tablets Prepared From Granules Prepared Using High-Shear Granulation With Normal Moisture Level (Regularly Granulated)

Hypromellose was dissolved in purified water and sprayed into a mixture of rivaroxaban, lactose monohydrate, microcrystalline cellulose, croscarmellose sodium, and sodium lauryl sulfate in a high-shear granulator. Additional water was added to an endpoint moisture of 34%. The wet granules were dried in a fluid bed drier and then milled through a Fitzmill with #1A screen. The milled granules were blended with the extra-granular components in a blender and the resulting blend was compressed into tablets on a rotary press.

### Example 3

### Formulation and Performance of Rivaroxaban Tablets, 10 mg, From Examples 1 & 2

The granules prepared according to Examples 1 and 2 were analyzed for their final blend bulk density (FB BD) and final blend particle size distribution (FB PSD). These results are in Table 1, below. Notably, Example 2, which uses conventional high-shear granulation techniques, results in larger particles (undesirable), but a higher-density product (desirable).

TABLE 1
Formulation and Performance of
Rivaroxaban Tablets, 10 mg-Examples 1 & 2

| | Example 1 | | Example 2 | |
|---|---|---|---|---|
| Ingredients | mg/tablet | % w/w | mg/tablet | % w/w |

Granulating Solution

| | | | | |
|---|---|---|---|---|
| Hypromellose | 0.82 | 1.0 | 0.82 | 1.0 |
| Purified Water¹ | (15.6) | N/A | (19.9) | N/A |

Intragranular Components

| | | | | |
|---|---|---|---|---|
| Rivaroxaban | 10.0 | 12.2 | 10.0 | 12.2 |
| Microcrystalline Cellulose | 32.8 | 40.0 | 32.8 | 40.0 |
| Lactose Monohydrate | 32.64 | 39.8 | 32.64 | 39.8 |
| Croscarmellose Sodium | 2.46 | 3.0 | 2.46 | 3.0 |
| Sodium Lauryl Sulfate | 0.82 | 1.0 | 0.82 | 1.0 |

Extragranular Components

| | | | | |
|---|---|---|---|---|
| Croscarmellose Sodium | 1.64 | 2.0 | 1.64 | 2.0 |
| Magnesium Stearate | 0.82 | 1.0 | 0.82 | 1.0 |
| Core Tablet Weight | 82.0 | 100.0 | 82.0 | 100.0 |

Characteristics

| Granulation endpoint | | finely granulated | regularly granulated |
|---|---|---|---|
| observation | | | |
| FB BD (g/mL) | | 0.43/0.57 | 0.64/0.72 |
| (poured/tapped) | | | |
| FB PSD | on #40 mesh | 0 | 9.0 |
| (%) | screen | | |
| | on #60 mesh | 0 | 12.3 |
| | screen | | |
| | on #80 mesh | 1.6 | 20.5 |
| | screen | | |
| | on #100 mesh | 1.2 | 13.1 |
| | screen | | |
| | on #140 mesh | 8.3 | 23.4 |
| | screen | | |
| | in pan | 88.9 | 21.7 |

### Example 4

### Dissolution Profile of Tablets Prepared According to Examples 1 and 2 Compared to XARELTO®

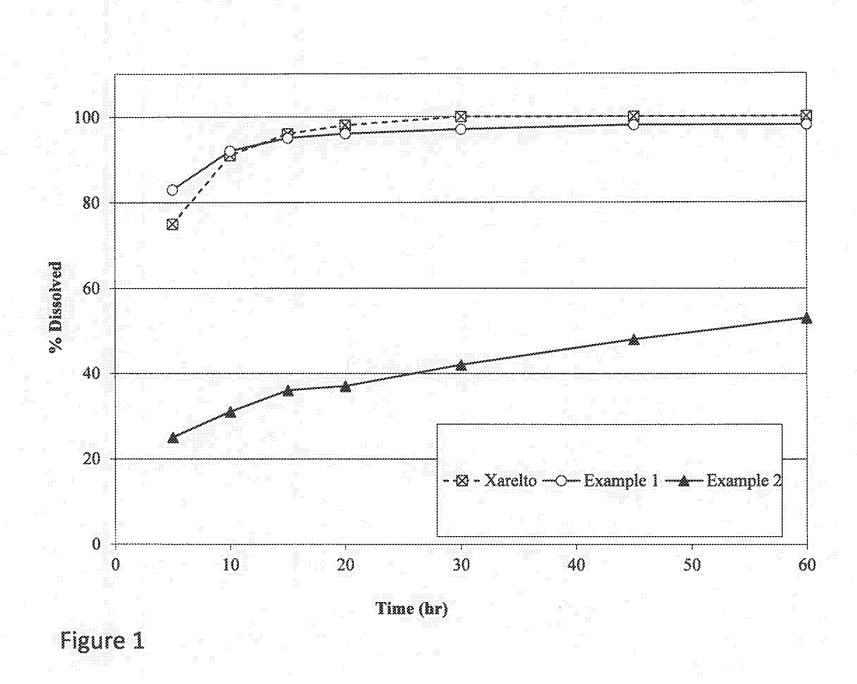

The dissolution profiles for tablets prepared according to Examples 1 and 2 were obtained in pH 4.5 acetate buffer with 0.2% SLS using USP Apparatus 2 (paddles) at 75 rpm. Notably, tablets prepared from the smaller-sized granules achieved from fluid bed granulation (Example 1) dissolve at a rate comparable to XARELTO® whereas tablets prepared from the larger granules prepared using conventional high-shear granulation (Example 2) dissolve at a much slower rate. Representative dissolution data are presented in FIG. 1.

### Example 5

### Formulation of Tablets Using Granules Prepared Using High-Shear Granulation With Low Moisture Level and Extended Time (High-Shear, Finely Granulated)

Hypromellose was dissolved in purified water and sprayed into a mixture of rivaroxaban, lactose monohydrate, microcrystalline cellulose, croscarmellose sodium, and sodium lauryl sulfate in a high-shear granulator with a target endpoint moisture of 25%. After the binder solution was consumed, the granulation was continued by mixing for 3 minutes extended time. The wet granulation was dried in a fluid bed drier and then milled through a Fitzmill with #1 screen. The milled granulation was blended with the extra-granular components in a blender and the blend was compressed into tablets on a rotary press. The final blend bulk density (FB BD) and final blend particle size distribution (FB PSD) for the tablets were determined. These results are in Table 2, below. Notably, low moisture, high-shear granulation results in granules with a bulk density higher than that achieved by fluid bed granulation (Example 1, Table 1) and granules which are smaller and have a narrower particle size distribution than those achieved by conventional high-shear granulation (Example 2, Table 1).

TABLE 2
Formulation and Performance of
Rivaroxaban Tablets, 10 mg-Example 5

| | Example 5 | |
|---|---|---|
| Ingredients | mg/tablet | % w/w |

Granulating Solution

| | | |
|---|---|---|
| Hypromellose (6 cP) | 2.40 | 3.0 |
| Purified Water¹ | (25.9) | N/A |

Intragranular Components

| | | |
|---|---|---|
| Rivaroxaban | 10.0 | 12.5 |
| Microcrystalline Cellulose (101) | 40.24 | 50.3 |
| Lactose Monohydrate (Impalpable) | 21.76 | 27.2 |
| Croscarmellose Sodium | 2.40 | 3.0 |
| Sodium Lauryl Sulfate | 0.80 | 1.0 |

Extragranular Components

| | | |
|---|---|---|
| Croscarmellose Sodium | 1.60 | 2.0 |
| Magnesium Stearate | 0.80 | 1.0 |
| Core Tablet Weight | 80.0 | 100.0 |

Characteristics

| Granulation endpoint observation | | finely granulated |
|---|---|---|
| FB BD (g/mL) (poured/tapped) | | 0.54/0.67 |
| FB PSD (%) | on #40 mesh screen | 5.5 |
| | on #60 mesh screen | 15.3 |
| | on #80 mesh screen | 8.5 |
| | on #100 mesh screen | 1.7 |
| | on #140 mesh screen | 6.4 |
| | in pan | 62.6 |

### Example 6

### Dissolution Profile of Tablets Prepared by Example 5 (Low Moisture High-Shear Granulation) Compared to XARELTO®

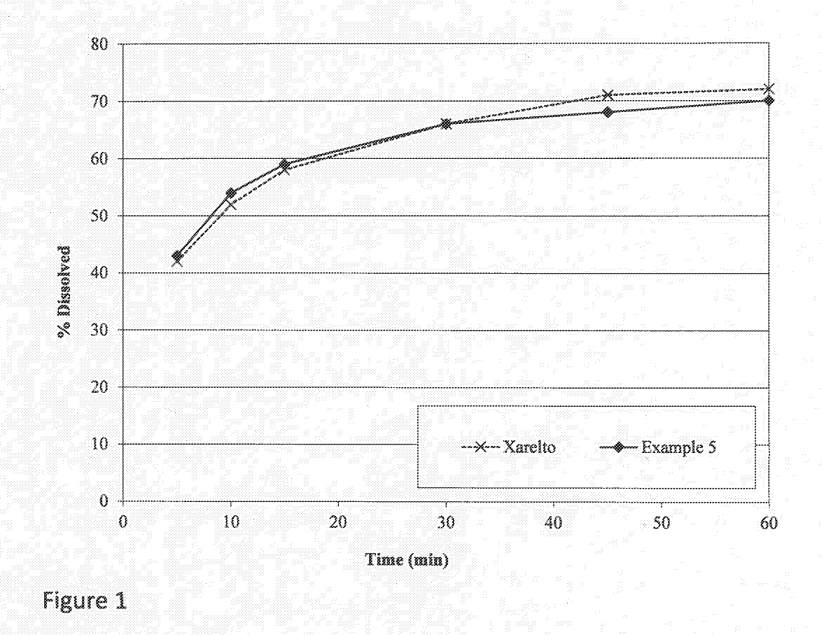

The dissolution profile for the tablets prepared by low moisture high-shear granulation (Example 3) was obtained in pH 4.5 acetate buffer with 0.2% SLS using USP Apparatus 2 (paddles) at 75 rpm. Those tablets prepared by low-moisture high-shear granulation dissolve at a rate comparable to XARELTO®. Notably, this is a faster dissolution rate than the dissolution rate of those granules prepared using conventional high-shear granulation (Example 2; see. FIG. 1). Representative dissolution data are presented in FIG. 2.

### Example 7

### Formulation of Tablets Using Granules Prepared Using High-Shear Granulation With Low Moisture Level and Extended Mixing Time (Finely Granulated)

The tablets of Example 7 were prepared using a similar procedure as disclosed in Example 5.

### Example 8

### Formulation of Tablets Using Granules Prepared With Fluid Bed Granulation

The tablets of Example 8 were prepared using a similar procedure as disclosed in Example 1.

### Example 9

### Formulation and Performance of Rivaroxaban Tablets (20 mg) From Examples 7 & 8

The tablets prepared according to Examples 7 and 8 were analyzed for their bulk density (BD) and particle size distribution (PSD). These results are shown in Table 3 below. Also, dissolution of these tablets were analyzed in comparison with XARELTO® (FIG. 2). Notably, granules prepared using high-shear granulation with low-moisture and extended mixing time (Example 7) have a similar (small) particle sizes and similar (narrow) particle size distributions as those prepared by fluidized bed granulation (Example 8), but with the desired higher bulk density typically not achieved in fluidized bed granulation. The bulk density is quite close to the high bulk density achieved in conventional high-shear granulation (Example 1). The dissolution of the tablets from Example 7 and Example 8 are also similar to each other and to XARELTO®.

TABLE 3
Formulation and Performance of
Rivaroxaban Tablets, 20 mg-Examples 7 & 8

| | Example 7 | | Example 8 | |
|---|---|---|---|---|
| Ingredients | mg/tablet | % w/w | mg/tablet | % w/w |

Granulating Solution

| | | | | |
|---|---|---|---|---|
| Hypromellose (6 cP) | 2.40 | 3.00 | 2.40 | 5.00 |
| Purified Water¹ | (25.9) | N/A | (25.7) | N/A |

Intragranular Components

| | | | | |
|---|---|---|---|---|
| Rivaroxaban | 20.0 | 25.0 | 20.0 | 25.0 |
| Microcrystalline Cellulose | 30.84 | 38.6 | 30.6 | 38.25 |
| (101) | | | | |
| Lactose Monohydrate | 21.76 | 27.2 | 21.0 | 26.25 |
| (Impalpable) | | | | |
| Croscarmellose Sodium | 2.40 | 3.0 | 2.40 | 3.0 |
| Sodium Lauryl Sulfate | 0.40 | 0.5 | 0.80 | 1.0 |

Extragranular Components

| | | | | |
|---|---|---|---|---|
| Croscarmellose Sodium | 1.60 | 2.0 | 1.60 | 2.0 |
| Magnesium Stearate | 0.40 | 0.5 | 0.80 | 1.0 |
| Yellow Iron Oxide | 0.20 | 0.25 | 0.20 | 0.25 |
| Colloidal Silicon Dioxide | — | — | 0.20 | 0.25 |
| Core Tablet Weight | 80.0 | 100.0 | 80.0 | 100.0 |
| White Opadry II (85F18422) | — | — | — | — |
| Beige Opadry II (85F170012) | 4.0 | 5.0² | 4.0 | 5.0² |
| Film Coated Tablet Weight | 84.0 | 105.0 | 84.0 | 105.0 |

Characteristics

| Granulation endpoint | | finely granulated | finely granulated |
|---|---|---|---|
| observation | | | |
| FB BD (g/mL) | | 0.58/0.73 | 0.42/0.53 |
| (poured/tapped) | | | |
| FBPSD (%) | on #40 mesh | 8.7 | 0.4 |
| | screen | | |
| | on #60 mesh | 17.9 | 2.4 |
| | screen | | |
| | on #80 mesh | 8.3 | 9.9 |
| | screen | | |
| | on #100 mesh | 4.8 | 9.1 |
| | screen | | |
| | on #140 mesh | 5.6 | 21.3 |
| | screen | | |
| | in pan | 54.8 | 56.9 |

### Example 10

### Bioequivalence Studies

Tablets formulated from granules prepared using low moisture, high-shear granulation techniques (Example 7) were found to be bioequivalent to XARELTO® and tablets formulated from granules prepared using fluidized bed granulation. The 3-way in vivo study was conducted under both fasting and fed conditions. The bioequivalence results (as measured by Cₘₐₓ and AUC_{inf}) for Examples 7 and 8 are in Table 4. These values are reported as a % of corresponding pharmacokinetic value for XARELTO® under identical conditions. Notably, tablets prepared from granules prepared using low moisture, high-shear granulation with extended mixing times (Example 7) have a similar bioavailability to XARELTO® and to the granules prepared using fluidized bed granulation (Example 8), illustrating that the innovative high-shear granulated formulation was as good as the fluid bed granulated formulation.

TABLE 4
3-Way In Vivo Bioequivalence Study Results¹ of
Rivaroxaban Tablets, 20 mg, Made from Innovative High
Shear Granulation Process (Example 7) and Fluid
Bed Granulation Process (Example 8) versus Xarelto ® 20 mg

| | Fasting (n = 15) | | Fed (n = 14) | |
|---|---|---|---|---|
| Lot No. | Cₘₐₓ | AUCinf | Cₘₐₓ | AUCinf |
| Example 7 | 100 | 105 | 96 | 101 |
| (High Shear Granulated) | (87-115) | (92-118) | (88-105) | (96-107) |
| Example 8 | 95 | 91* | 94 | 95 |
| (Fluid Bed Granulated) | (83-109) | (81-104) | (87-104) | (90-101) |

¹The data is expressed as mean (90% confidence interval) of % test/reference ratio.
*N = 14

1. A process for the preparation of rivaroxaban-containing granules, comprising the steps of:
a. dry mixing rivaroxaban and a pharmaceutically acceptable excipient in a high-shear mixer to form a dry mix blend;
b. adding a binder solution to the dry mix blend to form a granulating mixture;
c. mixing the granulating mixture in a high-shear mixer to form the rivaroxaban-containing granules; and
d. drying and milling the rivaroxaban-containing granules.

2. The process according to claim 1, wherein the binder solution comprises a binder dissolved in a solvent.

3. The process according to claim 2, wherein the binder is selected from the group consisting of hypromellose, cellulose or cellulose derivatives, povidone, starch, sucrose, polyethylene glycol, and mixtures thereof.

4. The process according to claim 2, wherein the solvent is selected from the group consisting of water, C₁₋₆ alcohol, and mixtures thereof.

5. The process according to claim 1, wherein the pharmaceutically acceptable excipient is selected from the group consisting of a lubricant, a glidant, a disintegrant, a bulking agent, a rate-controlling polymer, a filler, a surfactant, and mixtures thereof.

6. The process according to claim 1, wherein the granulating mixture has a water content of less than 30% and wherein the mixing step c. is carried out for between three minutes and five minutes.

7. A population of granules comprising rivaroxaban and a pharmaceutically acceptable excipient, wherein at least 50% of the population of granules have a diameter of less than 0.105 mm.

8. A solid pharmaceutical dosage form comprising the population of granules of claim 7.
